(19) United States
(12) Patent Application Publication
(10) Pub. No.: US 2016/0214912 A1
(43) Pub. Date: Jul. 28, 2016

(54) HYDROGEN ABSTRACTION FROM ALKANES USING HYDROGEN STORAGE MATERIALS

(71) Applicants: Scott F. MITCHELL, Sugar Land, TX (US); Travis R. CONANT, Sugar Land, TX (US); Lars C. GRABOW, Sugar Land, TX (US); SABIC GLOBAL TECHNOLOGIES B.V., Bergen Op Zoom (NL)

(72) Inventors: Scott F. Mitchell, The Woodlands, TX (US); Travis R. Conant, Richmond, TX (US); Lars C. Grabow, Houston, TX (US)

(21) Appl. No.: 14/899,292

(22) PCT Filed: Jul. 15, 2015
(86) PCT No.: PCT/US2015/040599

§ 371 Date: Dec. 17, 2015

Related U.S. Application Data
(60) Provisional application No. 62/026,124, filed on Jul. 18, 2014.

(51) Int. Cl. C07C 2/76

(52) U.S. Cl. CPC C07C 2/76

(57) ABSTRACT

Disclosed is a method for making C₂ or larger hydrocarbons from methane or for dehydrogenating C₂ or larger hydrocarbons. The method can include contacting methane or C₂ or larger hydrocarbons with a hydridable material under reaction conditions sufficient to effect removal of at least one hydrogen atom from a plurality of methane molecules or C₂ or larger hydrocarbons to produce a plurality of methyl radicals or to dehydrogenate the C₂ or larger hydrocarbons. With respect to the produced plurality of methyl radicals, they can combine together to form C₂ or larger hydrocarbons. The reaction is performed in the absence of oxygen gas and reactive metal oxides.

### CROSS REFERENCE TO RELATED APPLICATIONS

This application claims benefit to U.S. Provisional Patent Application No. 62/026,124 titled “HYDROGEN ABSTRACTION FROM ALKANES USING HYDROGEN STORAGE MATERIALS,” filed Jul. 18, 2014. The entire contents of the referenced application are incorporated herein by reference.

### BACKGROUND OF THE INVENTION

A. Field of the Invention

The invention generally concerns the production of high hydrocarbons from methane. In particular, a hydridable material (e.g., a metal) can be used to directly break C—H bonds and adsorb the hydrogen to form a hydride (e.g., metal hydride), with the resulting methyl radicals combining to form C₂ and larger hydrocarbons. Alternatively, C₂ and larger alkanes can be directly dehydrogenated to their corresponding alkenes (e.g., ethane to ethylene) with the hydridable material. These reaction processes can be performed without the assistance of oxygen gas and reactive metal oxides.

B. Description of Related Art

Methane is a chemically stable molecule due to the presence of its four strong tetrahedral C—H bonds (435 kJ/mol). Further adding to its stability is the fact that methane has no functional groups, magnetic moments, or polar distributions to undergo chemical attack. While there is an abundance of methane in today's society, its stability limits its use in efficiently converting methane into petrochemical feedstocks and liquid fuels.

The solutions currently offered in the petrochemical field to overcome methane stability include the use of high temperatures, the presence of oxygen, and/or the use of catalysts to activate methane into methyl radicals. Each of these solutions adds to the costs and complexity in the methane conversion process. By way of example, higher temperatures (i.e., 600 C.° and higher) not only increases the energy input, and thereby costs, in converting methane, it also increases the production of undesired side-products. In particular, oxidative coupling of methane (OCM) requires the use of high temperatures in the presence of O₂, which produces ethylene and carbon oxide (COₓ) side reactions.

One attempt to overcome the issues associated with the OCM reaction process was to use metal oxides rather than O₂ (see Xu et al., “Methane Activation by Transition-Metal Oxides, MO_{X} (M=Cr, Mo, W; x=1, 2, 3”, J. Phys. Chem A, 2002, Vol. 106, pp. 7171-7176). This proposed process, however, also required the use of a HZSM-5 catalyst. Further, the presence of oxygen resulted in unwanted side-products such as metal hydride compounds (H-MOₓ) and metal hydroxyl compounds (MOₓ—H), thereby complicating the possibility of regenerating the metal oxide from said compounds.

In U.S. Pat. No. 4,675,465, metals and metal alloys were used in combination with a metal oxide catalyst (Pt/Al₂O₃) to convert C₂ and higher alkanes to their corresponding alkenes. The metal oxide catalyst was used to break the C—H bond and reversibly bind H (i.e., catalytic cycle) at conditions required for methane activation, while the metals and metal alloys were used to adsorb the hydrogen, thereby adding to the costs and complexity of its process.

### SUMMARY OF THE INVENTION

A solution to the current problems associated with producing higher hydrocarbons from methane has been discovered. In particular the solution resides in contacting methane with a hydridable material to produce hydrocarbons having a carbon number of 2 or more. It was discovered that this reaction can be performed under relatively low temperature and pressure conditions without the assistance of oxygen gas and reactive metal oxides. Further, no catalysts are needed for this reaction, thereby allowing for stoichiometric or sub-stoichiometric reactions to occur. Therefore, the methods of the present invention have the benefit of efficiently producing economically valuable chemical compounds, examples of which include light olefins and paraffins, from methane. Still further, it was discovered that C₂ and larger alkanes can be converted to their corresponding alkenes under similar reaction conditions, thereby providing a solution for the deficiencies currently involved with such dehydrogenation reactions.

In one aspect of the invention, a method for making hydrocarbons (for example, C₂ hydrocarbons or higher) from methane includes contacting methane with a hydridable material under reaction conditions sufficient to effect removal of at least one hydrogen atom from a plurality of methane molecules to produce a plurality of methyl radicals in the absence of oxygen gas and reactive metal oxides. The plurality of methyl radicals combine together to ethane or ethylene. The reaction can continue as long as there is sufficient hydridable material to promote formation of alkyl radicals. The reaction to produce ethane from methane can be represented by reaction equation (I):

2 CH₄+XM→C₂H₆+XMH_{y}, (I)
where 0.05≧X≦1, M is the hydridable material, and y is 2/X.
   The reaction to produce ethylene from methane can be represented by reaction equation (II):

2 CH₄+XM→C₂H₄+XMH_{y}, (II)
where 1≧X≦4, M is the hydridable material, and y is 4/X.
   The hydridable material used in the invention is capable of exhibiting a negative free energy on hydride formation. In some instances, heat of formation of the hydridable material to the hydride is −64 kJ/mol (H₂) to −244 kJ/mol (H₂). In some aspects of the invention, the hydridable material is a metal, metal alloy, or an intermetallic compound. Non-limiting metals that can be used as a hydridable material include lithium (Li), sodium (Na), potassium (K), Rubidium (Rb), cesium (Cs), magnesium (Mg), calcium (Ca), strontium (Sr), barium (Ba), scandium (Sc), yttrium (Y), lanthanum (La), titanium (Ti), zirconium (Zr), hafnium (Hf), vanadium (V), tantalum (Ta), cerium (Ce), thallium (Th), uranium (U), and gadolinium (Gd). In certain aspects of the invention, the hydridable material is a metal from Group IVB of the Periodic Table. Non-limiting examples of Group IVB metals are titanium, zirconium and hathium, with a preferred metal being titanium. In one aspect of the invention, the hydridable material may be a combination of titanium and a second metal or a titanium metal alloy, where the second material is deposited on the surface of the metal. Non-limiting examples, of the second metal include nickel, palladium, platinum, or any combination thereof. In a preferred embodiment, palladium is deposited on the surface of titanium. Non-limiting examples of metal alloys include alloys that contain Group HA metals, Group IVB metals, Group VIII metals, or Group IVA elements. Examples of Group IIA metals include Mg, Ca, Sr, and Ba. Examples of Group VIII metals include, but are not limited to, iron (Fe), cobalt (Co), nickel (Ni), Ruthenium (Ru), Rhodium (Rh), palladium (Pd) and platinum (Pt). Examples of Group IVA elements include, but are not limited to, germanium (Ge), silicon (Si), carbon (C), tin (Sn) and lead (Pb). Other examples, of alloys or intermetallic compounds include titanium and platinum compounds and titanium and palladium compounds. In some aspects of the invention, the hydridable material is dispersed on a support. The support can be a non-reactive metal oxide support.

In some aspects of the invention, the reaction is run at stoichiometric or sub-stoichiometric conditions to control the exothermic properties of the reaction or the type of products produced (for example, paraffins versus olefins). The reaction is performed in the absence of oxygen. Without wishing to be bound by theory, it is believed that the presence of oxygen promotes carbon-oxygen bond formation instead of carbon-carbon bond formation. The amount of oxygen present in the reaction is 1,000 ppm or less, 100 ppm or less, or 10 ppm. The reaction is also run in the absence of metal oxides. Without wishing to be bound by theory, it is believed metal oxides promote acid-base type reactions (for example, hydronium ion H⁺ removal) and carbon-oxygen bond formation. In one aspect of the invention, the method can be performed under conditions that include a temperature of about 175 to 600° C. and a pressure of 15 to 800 psia (about 0.1 to 5.5 MPa). In some aspects of the invention, the method for making C₂ or higher hydrocarbons from methane can be performed in a first reactor, the produced hydride material is regenerated into the hydridable material, and the regenerated hydridable material is used to react with the methane. The produced hydride can be regenerated by subjecting the produced hydride to regeneration conditions to regenerate the hydridable material. Regeneration conditions include a sufficient amount of heat, an oxidizing agent, or a combination of both. A non-limiting example, of an oxidizing agent is oxygen gas. When the oxidizing agent is oxygen, water is produced in addition to the hydridable material. In some instances, the oxidizing agent is oxygen and water is produced as a by-product. In some aspects of the invention, the produced hydride material can be stored (e.g., a hydrogen storage material) and processed at a later time to produce hydridable material. In some aspects of the inventions, the hydride material can be transferred from the first reactor into a second reactor and subjected to regeneration conditions to form the hydridable material, and water or hydrogen depending on the regeneration conditions. The regenerated hydridable material can then be transferred into the first reaction chamber to react with the methane.

In another aspect of the invention, a method for removing hydrogen from a hydrocarbon to produce olefins has been discovered. The method includes contacting a hydrocarbon with a hydridable material under reaction conditions sufficient to effect removal of at least one hydrogen atom from one of the hydrocarbons by the hydridable material to produce ethylene or C₂ and larger hydrocarbon alkenes. The removed hydrogen atoms combine with the hydridable material to form a hydride material. The reaction to produce olefins from when the hydrocarbon source is a C₂-C₄ hydrocarbon can be represented by reaction equation (III).

CₙH₂ₙ₊₂+XM→CₙH_{2n+2-y}+XMH_{y/X}, (III)
where n is from 2 to 4, y is 2 or 4, X is ≧1, and M is the hydridable material.
   In some instances, n is 2 to 3, y is 2, and X is 1. In another instance n is 4, y is 4 and X is 1, 2 or 4. The produced products are C₂-C₄ olefins (for example, ethylene, propylene and butene), and in some instances, di-olefins (for example, butadiene). The reaction conditions, types of hydridable material used, types of reactors used, and types of reactions that occur in the reactor can be the same as those described directly above and throughout the specification.

In the context of the present invention the following 54 embodiments are described. Embodiment 1 is a method for making C₂ or larger hydrocarbons from methane. The method includes contacting methane with a hydridable material under reaction conditions sufficient to effect removal of at least one hydrogen atom from a plurality of methane molecules to produce a plurality of methyl radicals, wherein the plurality of methyl radicals combine together to form C₂ or larger hydrocarbons, and wherein the reaction is performed in the absence of oxygen gas and reactive metal oxides. Embodiment 2 is the method of embodiment 1, wherein ethane is produced and the overall reaction is represented by reaction (I):

2 CH₄+XM→C₂H₆+XMH_{y},
where 0.05≧X≦1, M is the hydridable material, and y is 2/X.
   Embodiment 3 is the method of embodiment 1, wherein ethylene is produced and the overall reaction is represented by reaction (II):

2 CH₄+XM→C₂H₄+XMH_{y},
where 1≧X≦4, M is the hydridable material, and y is 4/X.
   Embodiment 4 is the method of any one of embodiments 1 to 3, wherein the formation energy of the hydridable material to the hydride is −101 to −244 kJ/mol (H₂). Embodiment 5 is the method of embodiment 4, wherein the hydridable material is a metal, a metal alloy, or an intermetallic compound. Embodiment 6 is the method of embodiment 5, wherein the hydridable material is a metal selected from the group consisting of Li, Na, K, Rb, Cs, Mg, Ca, Sr, Ba, Sc, Y, Ti, Zr, Hf, V, Ta, La, Ce, Th, U, and Gd. Embodiment 7 is the method of embodiment 6, wherein the metal is a Group IVB metal. Embodiment 8 is the method of embodiment 7, wherein the Group IVB metal is Ti. Embodiment 9 is the method of any one of embodiments 6 to 7, wherein a second metal is deposited on the surface of the metal. Embodiment 10 is the method of embodiment 9, wherein the second metal is a Group IIA metal, Pd, Pt, Ni, or any combination thereof. Embodiment 11 is the method of embodiment 10, wherein Pd is deposited on the surface of Ti. Embodiment 12 is the method of embodiment 5, wherein the hydridable material is a metal alloy comprising a Group HA metal, a Group IVB metal, a Group VIII metal, or a Group IVA. Embodiment 13 is the method of any one of embodiments 1 to 12, wherein the overall reaction is exothermic. Embodiment 14 is the method of any one of embodiments 1 to 13, wherein the reaction conditions include a temperature of 175° C. to 600° C. and a pressure of 15 psia to 800 psia. Embodiment 15 is the method of any one of embodiments 1 to 12, wherein the reaction is performed in the absence of oxygen. Embodiment 16 is the method of any one of embodiments 1 to 15, wherein the hydridable material is supported by a support. Embodiment 17 is the method of embodiment 16, wherein the support is a non-reactive metal oxide support. Embodiment 18 is the method of any one of embodiments 1 to 17, wherein the reaction is a stoichiometric or sub-stoichiometric reaction. Embodiment 19 is the method of any one of embodiments 1 to 17, wherein the reaction is performed in a first reactor, the produced hydride is regenerated into the hydridable material, and the regenerated hydridable material is used to react with the methane. Embodiment 20 is the method of embodiment 19, wherein the produced hydride is subjected to a sufficient amount of heat to regenerate the hydridable material. Embodiment 21 is the method of embodiment 19, wherein the produced hydride is subjected to an oxidizing agent to regenerate the hydridable material. Embodiment 22 is the method of embodiment 21, wherein the oxidizing agent is O₂ and water is produced as a by-product. Embodiment 23 is the method of any one of embodiments 18 to 22, wherein the reaction is performed in the first reactor and the regeneration of produced hydride to the regenerated hydridable material is performed in a second reactor. Embodiment 24 is the method of any one of embodiments 18 to 23, wherein the reaction is performed in the first reactor and the regeneration of produced hydride to the regenerated hydridable material is performed in the first reactor.

Embodiment 25 is a method for removing hydrogen from a hydrocarbon. The method includes contacting a hydrocarbon with a hydridable material under reaction conditions sufficient to effect removal of at least one hydrogen atom from the hydrocarbon by the hydridable material and form a hydride from the hydridable material and the removed hydrogen atom, wherein the reaction is performed in the absence of oxygen gas and reactive metal oxides. Embodiment 26 is the method of embodiment 25, wherein the hydrocarbon is a C₂-C₄ hydrocarbon and the reaction is represented by reaction (III):

CₙH₂ₙ₊₂+XM→CₙH_{2n+2-y}+XMH_{y/X},
where n is from 2 to 4, y is 2 or 4, X is ≧1, and M is the hydridable material.
   Embodiment 27 is the method of embodiment 26, where n is 2, y is 2, and X is 1 or 2. The method of embodiment 26, where n is 3, and y is 2, and X is 1 or 2. Embodiment 29 is the method of embodiment 26, where n is 4, and y is 2, and X is 1 or 2. Embodiment 30 is the method of embodiment 26, where n is 4, y is 4, and X is 1, 2, or 4. Embodiment 31 is the method of embodiment 26, wherein the hydrocarbon is methane and the overall reaction is represented by reaction (II):

2 CH₄+XM→C₂H₄+XMH_{y},
where 1≧X≦4, M is the hydridable material, and y is 4/X.
   Embodiment 32 is the method of any one of embodiments 25 to 31, wherein the formation energy of the hydridable material to the hydride is −64 to −244 kJ/mol (H₂). Embodiment 33 is the method of any one of embodiments 25 to 32, wherein the formation energy of the hydridable material to the hydride is −101 to −244 kJ/mol (H₂). Embodiment 34 is the method of embodiment 33, wherein the hydridable material is a metal, a metal alloy, or an intermetallic compound. Embodiment 35 is the method of embodiment 34, wherein the hydridable material is a metal selected from the group consisting of Li, Na, K, Rb, Cs, Mg, Ca, Sr, Ba, Sc, Y, Ti, Zr, Hf, V, Ta, La, Ce, Th, U, and Gd. Embodiment 36 is the method of embodiment 35, wherein the metal is a Group IVB metal. Embodiment 37 is the method of embodiment 36, wherein the Group IVB metal is Ti. Embodiment 38 is the method of any one of embodiments 36 to 37, wherein a second metal is deposited on the surface of the metal. Embodiment 39 is the method of embodiment 38, wherein the second metal is a Group IIA metal, Pd, Pt, Ni, or a combination thereof. Embodiment 40 is the method of embodiment 39, wherein Pd is deposited on the surface of Ti. Embodiment 41 is the method of embodiment 34, wherein the hydridable material is a metal alloy comprising a Group HA metal, a Group IVB metal, a Group VIII metal, or a Group IVA. Embodiment 42 is the method of any one of embodiments 25 to 41, wherein the overall reaction is exothermic. Embodiment 43 is the method of any one of embodiments 25 to 42, wherein the reaction conditions include a temperature of 175° C. to 600° C. and a pressure of 15 psia to 800 psia. Embodiment 44 is the method of any one of embodiments 25 to 43, wherein the reaction is performed in the absence of oxygen. Embodiment 45 is the method of any one of embodiments 25 to 44, wherein the hydridable material is supported by a support. Embodiment 46 is the method of embodiment 45, wherein the support is a non-reactive metal oxide support. Embodiment 47 is the method of any one of embodiments 25 to 46, wherein the reaction is a stoichiometric or sub-stoichiometric reaction. Embodiment 48 is the method of any one of embodiments 25 to 47, wherein the reaction is performed in a first reactor, the produced hydride material is regenerated into the hydridable material, and the regenerated hydridable material is used to react with the methane. Embodiment 49 is the method of embodiment 48, wherein the produced hydride material is subjected to a sufficient amount of heat to regenerate the hydridable material. Embodiment 50 is the method of embodiment 49, wherein hydrogen gas evolves from the hydride material. Embodiment 51 is the method of embodiment 50, wherein the produced hydride is subjected to an oxidizing agent to regenerate the hydridable material. Embodiment 52 is the method of embodiment 51, wherein the oxidizing agent is O₂ and water is produced as a by-product. Embodiment 53 is the method of any one of embodiments 47 to 52, wherein the reaction is performed in the first reactor and the regeneration of produced hydride to the regenerated hydridable material is performed in a second reactor. Embodiment 54 is the method of any one of embodiments 47 to 53, wherein the reaction is performed in the first reactor and the regeneration of produced hydride to the regenerated hydridable material is performed in the first reactor.

The following includes definitions of various terms and phrases used throughout this specification.

The use of the term “methane” throughout this specification refers to a composition that includes a plurality of methane molecules.

The phrases in the “absence of oxygen”, “in the absence of oxygen gas,” and “in the absence of reactive metal oxides” means that the total amount of reactive oxygen atoms present in the reaction are less than 10,000 ppm, preferably less than 5,000 ppm, more preferably less than 1,000 ppm, and most preferably less than 100 ppm.

“Reactive oxygen atoms” means oxygen gas and compounds comprising oxygen in which the oxygen atom reacts with methane or C₂ and larger hydrocarbons or radicals thereof during the processes of the present invention to produce carbon oxides.

“Non-reactive metal oxide support” means a support in which the oxygen sites of the metal oxide do not participate in the reaction. For example, a metal oxide support that is neutral or acidic in nature. Examples of non-reactive metal oxide supports include silicon dioxide or titanium dioxide.

The term “Groups” refers to the family of elements in the Chemical Abstracts Service (CAS) Version of the Periodic Table.

The terms “about” or “approximately” are defined as being close to as understood by one of ordinary skill in the art, and in one non-limiting embodiment the terms are defined to be within 10%, preferably within 5%, more preferably within 1%, and most preferably within 0.5%.

The term “substantially” and its variations are defined as being largely but not necessarily wholly what is specified as understood by one of ordinary skill in the art, and in one non-limiting embodiment substantially refers to ranges within 10%, within 5%, within 1%, or within 0.5%.

The terms “inhibiting” or “reducing” or “preventing” or “avoiding” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the word “a” or “an” when used in conjunction with the term “comprising” in the claims or the specification may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The methods of the present invention can “comprise,” “consist essentially of,” or “consist of” particular ingredients, components, compositions, etc. disclosed throughout the specification. With respect to the transitional phase “consisting essentially of,” in one non-limiting aspect, a basic and novel characteristic of the methods of the present invention is their ability to produce C₂ and larger hydrocarbons from methane and to dehydrogenate C₂ and larger hydrocarbon alkanes into corresponding alkenes in the absence of oxygen gas and reactive metal oxides.

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description.

### BRIEF DESCRIPTION OF THE DRAWINGS

FIG. 1 is a schematic of a system of the present invention the present invention that includes one reactor.

FIG. 2 is a schematic of a system of the present invention for production of C₂ or higher hydrocarbons that includes two reactors.

### DETAILED DESCRIPTION OF THE INVENTION

While the use of catalysts, some of which are hydridable materials to convert methane to other products is known, these processes are inefficient, require complex catalysts (for example, Pt/Al₂O₃ catalyst, a metallocene type catalyst, etc.) result in unwanted by-products. Other known processes, for example, oxidative coupling of methane, or steam cracking of higher hydrocarbons to produce ethylene consume large amounts of energy. These processes require high temperatures, expensive catalysts and remain largely inefficient.

The present discovery offers a solution to these problems by contacting methane with a hydridable material to produce C₂ or higher hydrocarbons and a hydride material without the assistance of oxygen and/or reactive metal oxides. The hydride material is formed from the coupling of a hydrogen atom removed from the methane and the non-catalytic hydridable material. The hydride material can be used as a source of hydrogen and/or water. Under certain conditions the hydride material can be regenerated to form the hydridable material. The method includes contacting a hydrocarbon with the hydridable material under conditions sufficient to effect the production of C₂ or higher olefins from methane and other alkanes. The advantages of using the hydridable material are at least two-fold. First, the reactions can be conducted at a lower reaction temperature as compared to current processes. Secondly, side reactions due to high heat and/or the presence of oxygen are minimized and/or avoided.

These and other non-limiting aspects of the present invention are discussed in further detail in the following sections.

### A. Reactants

The reactants in the present invention include hydrogen containing compounds that have a hydrogen capable of being removed by a hydridable material under the conditions of the present invention. In some instances, the hydrogen containing compounds include methane, ethane, propane, butane, or any combination thereof. The hydrocarbons of the present invention can be obtained from gas fields, natural gas, biomass, hydrocarbon gas mixtures from chemical processing, or any combination thereof. In one aspect of the invention, the hydrocarbon is methane. In some aspects of the invention, the hydridable material is a metal, metal alloy, or an intermetallic compound from Group IA, Group IIA, Group IIIB, Group VIB, Group VB, Group IIIA, and the lanthanides of the Periodic Table that have an enthalpy of hydride formation between −64 kJ/mol (H₂) to −244 kJ/mol (H₂). Non-limiting examples of metals that form hydrides in the desired enthalpy of hydride formation range are lithium (Li), sodium (Na), potassium (K), Rubidium (Rb), cesium (Cs), magnesium (Mg), calcium (Ca), strontium (Sr), barium (Ba), scandium (Sc), yttrium (Y), lanthanum (La), titanium (Ti), zirconium (Zr), hafnium (Hf), vanadium (V), tantalum (Ta), cerium (Ce), thallium (Th), uranium (U), and gadolinium (Gd). Hydridable material can be commercially obtained from, for example, Sigma Aldrich®, Alfa Aesar® A Johnson Matthey Company, Ward Hill, Mass., and American Elements Corporation, The Materials Science Company®, Los Angeles, Calif. The hydride material formed from hydridable materials containing Group IA metals include, but are not limited to, LiH, NaH, KH, RbH, CsH, or any combinations thereof. Non-limiting examples, of hydride material formed from hydridable material containing Group HA metals include MgH₂, CaH₂, SrH₂, BaH₂, or any combination thereof. Group IIIB hydride materials formed from hydridable materials containing Group IIIB metals include, but are not limited to, ScH₂, and YH₂. Group IVB hydride materials formed from hydridable materials containing Group IVB metals include, but are not limited to, TiH, TiH₂, TiH₄, ZrH₂, HfH, or any combination thereof. Titanium is a preferred hydridable material of the present invention as TiH₂ has an enthalpy of formation of −144.3 kJ/mol (H₂). A non-limiting example of a hydride material formed from hydridable material containing Group IIIA metals is AlH₃. Hydride materials formed from hydridable materials containing a lanthanide includes LaH₂, CeH₂, ThH₂, UH₃, GdH₂, or any combination thereof. In some aspects of the invention, the hydridable material is dispersed on a support. The support can be a non-reactive metal oxide support. Non-limiting examples, of non-reactive supports include silicon dioxide, carbon, and polymers.

In one aspect of the invention, the hydridable material may be a combination of titanium and a second metal or a titanium metal alloy, where the second material is deposited on the surface of the metal. Non-limiting examples of the second metal include nickel, palladium, platinum, or any combination thereof. In a preferred embodiment, palladium is deposited on the surface of a titanium metal, metal alloy or intermetallic compound. The second metal can be deposited on the first metal using pulsed laser deposition, vapor phase deposition, plasma-assisted deposition, or other suitable deposition methods. Metal alloys having an enthalpy of hydride formation between −64 to −244 kJ/mol (H₂) can be used in the present invention to make C₂ or higher hydrocarbons. Non-limiting examples of metal alloys include alloys that contain a metal from the Groups described throughout this specification, preferably from Groups HA and IVB, and another metal. Non-limiting examples of metal alloys that form hydrides of the present invention include MgNiBe, Mg₂Ni_{1-y}Be_{y}, where y is 0.15-0.25, Th_{1.5}Ce_{0.5} Al, Th₂Al, Ti₂Cu, Ti₂Pd, Zr₂Cu(M), Zr₂Ni, Mg_{1.92}Al_{0.08}Ni, Mg₂Co, Mg₂Co, Mg₆Co₂, Mg₂Cu, Mg₂Fe, Mg₂FeH₆, Mg₂Ni, Mg₂Ni_{0.75}Co_{0.25}, Mg₂Ni_{0.75}Fe_{0.25}, Mg_{0.833}Ni_{0.066}Cu_{0.095}M_{1.006}, Mg_{0.9}Sc_{0.1}, Mg_{0.708}La_{0.125}Al_{0.067}, Mg_{0.70}Ni_{0.25}Nd_{0.055} Mg_{0.8}Ni_{0.1}Al_{0.1}, Mg_{1.7}NiAl_{0.3}, Mg_{1.9}NiAl_{0.08}, Mg-10Ni, Mg-12Ce, Mg₁₄Al₁₂Ti₃, Mg₁₇Al₁₂, Mg_{0.85}La_{0.1}Al_{0.05}, Mg_{0.9}La_{0.075}Al_{0.025}, Mg₂CuAl_{0.375}, Mg₂Ni_{0.63}Fe_{0.37}, Mg₂Ni_{0.75}Fe_{0.25}, Mg₂Ni_{0.75}Al_{0.25}, Mg₂Ni_{0.85}Al_{0.15}, Mg₂Ni_{0.75}Co_{0.25}, TiCu, Ti₃Sb, Ti₃Sn, ErNi, LaNi, LaNi, Li_{0.94}Pd, LiPd, LiPt, LuNi, YbNi, YbPd, Zr_{0.5}Hf_{0.5}Co, Zr_{0.7}Hf_{0.3}Co, ZrCo, ZrCo_{0.84}Ni_{0.16}, ZrNi, CaNi₂, CaMg₂, Th_{1.5}Ce_{0.5}Al, UniAl, ZrV₂, GdMn₂, GdNi₂, LaNi₂, PrCo₂, CeFe₅, CeMg₅, LnMg₂, Ca₄Mg₄Fe₃H₂₂, Zr_{1-y}Al_{y}, where y=0.25-0.75, Zr₃V₃O, Zr_{0.975}Nb_{0.25}, Zr₁₋ₓHfₓ, where x=0.23-0.82, Ta_{0.4}Ti_{0.6}, Ta_{0.5}Ti_{0.5}, CsAlH₄, KAlH₄, KBH₄, LiBH₄, Yb₄Mg₄Fe₃H₂₂, Na₃AlH₆, Na₃BH₄, or any combination thereof.

### B. Hydrogen Removal

The hydridable material of the present invention is capable of removing a hydrogen atom from methane or other hydrocarbons (i.e., breaking a C—H bond) to form a hydride material from the extracted hydrogen atom and the hydridable material. The hydride material can be regenerated to from the hydridable material, hydrogen or water depending on the regeneration conditions and can be represented by the regeneration reaction equation (IV) shown below.

XMHy+A→XM−Y, (IV)
where X≧1, M is the hydridable material, y is 1 to 4, A is either heat or oxidant, and Y is hydrogen (H₂) or water.
   The reactions shown in Equations (I), (II) or (III) are exothermic when a heat of formation of the hydride material ranges between −64 and −244 kJ/mol (H₂), −100 and −200 kJ/mol (H₂), or −125 and −150 kJ/mol (H₂), therefore the overall reaction of equations (I), (II), or (III) in combination with equation (IV) is exothermic. The hydridable material under the conditions of the reaction removes at least one hydrogen atom from a plurality of methane molecules (for example, 2 methane molecules) to produce a plurality of methyl radicals, which combine to from a plurality of ethane molecules. Without wishing to be bound by theory, it is believed that the hydridable material has surface sites that are sufficiently active to adsorb hydrocarbon molecules, methyl radicals and hydrogen radicals. At least one C—H bond is broken on the surface of the hydridable material to produce a plurality of methyl radicals and hydrogen radicals, which are adsorbed on the surface of the hydridable material as illustrated in reaction equation (V), with * being the hydridable material.

CH₄+2*→CH₃*+H* (V)

Two surface bound methane radicals can combine in various manners. In a first manner, the methyl radicals can directly combine to form ethane and the ethane desorbs from the surface of the hydridable material, which produced two free active surface sites on the hydridable material as illustrated in reaction equation (VI).

2 CH₃*→C₂H₆+2* (VI)

In a second manner, two methyl radicals can combine to form ethane, which is initially adsorbed on the surface of the hydridable material and then desorb to produce two active surface sites as illustrated in reaction equation (VII).

2 CH₃*→C₂H₆*+*→C₂H₆+2* (VII)

In a third manner two methyl radicals desorb from the active surface sites of the hydridable material and form ethane in the gas phase as illustrated in reaction equation (VIII).

2 CH₃*→2 CH₃+2*→C₂H₆+2* (VIII)

The surface bound hydrogen (xH*) diffuse into the bulk of the hydridable material and forms the hydride material MHx and an active surface site on the hydridable material as illustrated in reaction equation (IX).

xH*+M→MHₓ+* (IX)

Without wishing to be bound by theory, it is also believed that the hydridable material can remove two hydrogen radicals from the methane molecules in a step wise fashion to form methyl diradicals (carbene type molecules CH₂:) that are adsorbed on the surface of the hydridable material as illustrated in reaction equation (X). The second hydrogen is abstracted on the catalytically active surface site to form the surface bound carbene (CH₂*).

CH₄+2*→CH₃*+H*

CH₃*+*→CH₂*+H* (X)

Two or more carbenes can combine to form ethylene in a various manners. In one manner, two surface bound carbenes can directly from ethylene and restore the active sites on the hydridable material as shown in reaction equation (XI).

2 CH₂*→C₂H₄+2* (XI)

In a second manner, two surface bound carbenes can from ethylene, which is initially adsorbed. Desorption of ethylene after a period of time frees up active surface sites on the hydridable material as shown in reaction equation (XII).

2 CH₂*→C₂H₄*+*→C₂H₄+2* (XII)

In a third manner, surface bound carbenes desorb to from free carbene radicals and active surface sites on the hydridable material. The carbenes combine in a homogeneous gas-phase reaction to form ethylene as shown in reaction equation (XIII).

2 CH₂*2 CH₂+2*→C₂H₄ (XIII)

As previously described in reaction equation (IX), the surface bound hydrogen (xH*) from reaction equation (X) can diffuse into the bulk of the hydridable material and form the hydride material MHx and an active surface site on the hydridable material. It should be understood that reaction mechanism equations (V) through (XIII) are for illustrative purposes only as other reactive intermediates may be envisioned.

In another aspect of the invention, C₂ or higher olefins can be produced, for example, from methane, ethane, propane, butane, or any combination thereof. Similar to the reaction of the hydridable material with methane, a hydride material is produced where the hydrogen is obtained from the starting hydrocarbon as shown in reaction equation (III). Without wishing to be bound by theory, it is believed that when a C₂ or higher hydrocarbon is the reactant, the hydrocarbon adsorbs on the surface of the hydridable material and the C—H bond is broken to form a hydrocarbon radical and a hydrogen radical, both of which are adsorbed on the surface of the hydridable material as illustrated for propane in reaction equation (XIV).

C₃H₈+2*→C₃H₇*+H* (XIV)

A hydrogen atom can be abstracted by an active site on the hydridable material to form propene and a hydrogen radical, which are adsorbed on the surface of the hydridable material as shown in reaction equation (XV).

C₃H₇*+*C₃H₆*+H* (XV)

The propylene can desorb from the hydridable material to free up an active surface site on the hydridable material as shown in reaction equation (XVI).

C₃H₆*→C₃H₆+* (XVI)

The surface bound hydrogen radicals can diffuse into the bulk of the hydridable material and form the hydride material MHx and an active surface site on the hydridable material as shown in reaction equation (IX). The reaction can continue until a majority, substantially all, or all of the active surface sites of the hydridable material contain hydrogen, thereby, exhausting the hydrogen storage capability of the hydridable material.

### C. One and Two Reactor Processes

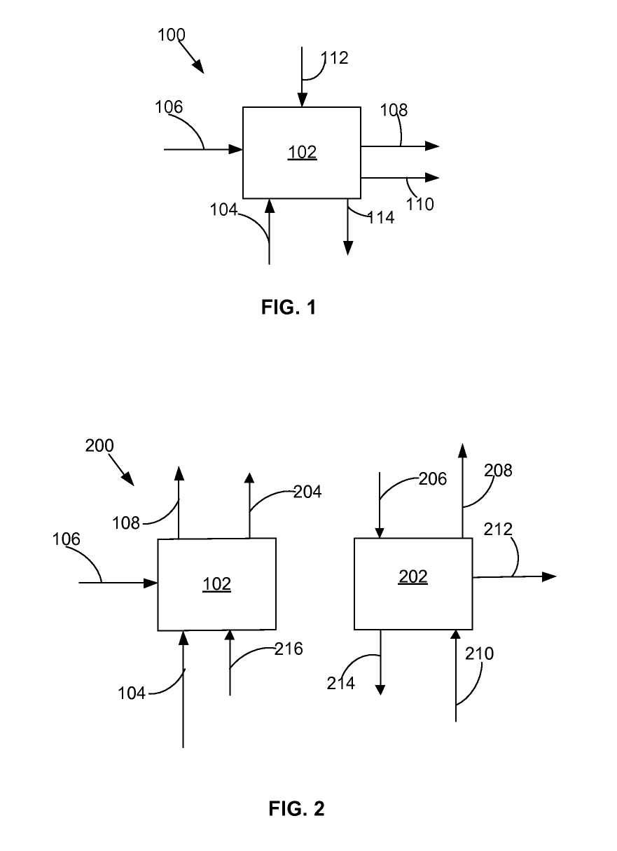

One or two reactors can be used in the context of the present invention to treat methane or C₂-C₄ hydrocarbons with a hydridable material to produce ethane and/or olefins. A hydrocarbon feed (for example, methane, natural gas, or C₂-C₄ hydrocarbons) is fed to a reactor. The hydridable material is contacted with the hydrocarbon feed at temperatures and pressures suitable to form a hydride material that includes hydrogen atoms extracted from the hydrocarbon to produce ethane or C₂-C₄ olefins depending on the starting material (for example, ethylene is made from methane or ethane, propene is made from propane, etc.). The produced hydrocarbons can be removed from the reactor and the hydride material can be regenerated to form the hydridable material. Non-limiting examples of a single reactor (FIG. 1) and a two reactor (FIG. 2) processes are provided below.

Referring to FIG. 1, a schematic of system 100 to produce C₂ or higher hydrocarbons can include a reactor 102 that is configured to produce product (C₂ or higher hydrocarbons) and regenerate the hydride material. Examples of reactors that can be used in the context of the present invention include fluidized bed reactors, fixed bed reactors, transport bed reactors, ebullating bed reactors, slurry reactors, rotating kiln reactors, continuously stirred tank reactors, spray reactors, or gas/solid contactors. Reactor 102 includes suitable heating and cooling elements known in the art, for example, electrical heaters, heat exchangers, cooling jackets, etc. The reactor may be equipped with inert gas inlets to allow the reactor to be charged and/or operated under an inert atmosphere. Operating under an inert atmosphere lessens and/or inhibits oxygen from the atmosphere from entering the reactor 102. The hydridable material may be charged or fed into the reactor 102 via the hydridable material inlet 104. Non-limiting examples of hydridable material that can be used in the context of the present invention are the hydridable materials described throughout this specification. The hydrocarbon feed enters the reactor 102 via the hydrocarbon feed inlet 106. The hydridable material and the hydrocarbon feed can be charged at the same or different times to the reactor 102. Contact of the hydrocarbon feed with the hydridable material under an inert atmosphere and without the assistance of a reactive metal oxide produces a hydrocarbon product of the present invention (for example, C₂ or higher hydrocarbons such as, but not limited to, ethane, ethylene, propylene, butene, isobutene, or any combination thereof) and a hydride material that contains at least one hydrogen from the reactant hydrocarbon feed. The hydrocarbon product can exit the reactor 102 via a product outlet 108. In some instances, the product is produced as a vapor. The produced product can be stored, sold commercially, or transported to other processing units for conversion into high value chemical products.

The produced hydride material, generally being a solid or having a higher boiling point than the hydrocarbon product, can remain in the reactor 102. Once sufficient, or substantially all, hydrocarbon product is removed from the reactor 102, the reactor can be heated to a temperature sufficient to regenerate the hydridable material through the evolution of hydrogen from the hydride material. The hydrogen can be removed from the reactor 102 via the hydrogen outlet 110, and captured for use in other chemical processes and/or for energy production. Alternatively, the hydride material can be treated with an oxidant (for example, air, oxygen enriched air, or oxygen gas) at conditions sufficient to produce water and the hydridable material from the hydride material. For example, oxidant can enter the reactor 102 via the oxidant inlet 112. If necessary, heat can be provided during the addition of oxidant. In some instances, the reaction is sufficiently exothermic that no external heat is necessary. The regeneration temperature can be controlled through heat exchange with a heat transfer fluid coupled to the reactor 102. The produced water can be removed from the reactor 102 through the water outlet 114 or physically separated from the hydride material using known solid/liquid or gas/liquid separation techniques (for example, centrifugation, filtering, etc.). After sufficient hydridable material is formed, additional hydrocarbon feed can be provided to the reactor 102 through the hydrocarbon feed inlet 106 and the process can be continued. In some instances, system 100 can be automated to allow feed and, in some instances hydridable material or oxidant, to be provided to the reactor 102 based on the composition of the streams exiting the reactor 102. The hydridable feed inlet 104, the hydrocarbon feed inlet 106, the oxidant inlet 112, the product outlet 108, the hydrogen outlet 110 and/or the water outlet 114 can be automated or semi-automated such that the production of product and regeneration of hydride is a continuous process. For example, the hydrocarbon feed inlet 106, the hydrocarbon product outlet 108, the oxidant inlet 112, the hydrogen outlet 110 and the water outlet 114 can be connected through an electronic feedback loop (for example, a computer system and/or control system) and sensors. The sensors can determine when product production is diminished and/or ended, and regulate the feed inlet 106 to terminate or temporarily stop the flow of feed into the reactor. Once a majority or substantially all of the hydrocarbon product has exited through the product outlet 108, the automated system can adjust the temperature to heat the reactor 102 to a temperature sufficient to produce a regenerated hydridable material. In the instances when oxygen is used, the automated system can open the oxidant inlet 112 and provide oxidant to the reactor 102 and control the temperature and pressure for the hydridable material regeneration process. Once water production has diminished or stopped, the automated system can provide a signal to close the oxidant inlet 112 and open the feed inlet 106 to continue the process. In some instances, the reactor 102 is flushed with inert gas to remove any traces of oxygen from the system prior to opening the feed inlet 106.

Referring to FIG. 2, a schematic of system 200 for production of C₂ or higher hydrocarbons that can include a reactor 102 (such as the reactor 102 in System 100) and a second reactor 202 is described. The second reactor 202 can be used to regenerate the hydride material. Reactor 202 can be a batch reactor, a continuous reactor, or the like. In the reactor 102, contact of the hydrocarbon feed with the hydridable material produces a hydride material and a hydrocarbon product of the present invention. The hydrocarbon product can exit reactor 102 via hydrocarbon product outlet 108. The produced hydrocarbon product can be stored, sold commercially, or transported to other processing units for conversion into high value chemical products. During the course of the reaction, or at the end of the reaction period, the hydride material can exit the reactor 102 via hydride material outlet 204. The hydride material can be stored and treated at a later time to produce hydridable material, and hydrogen or water. In some instances, the hydride material has a different boiling point than the produced hydrocarbons and is separated from the hydrocarbons and hydride material as a vapor stream via hydride material outlet 204. In some instances, the hydride material is removed from the reactor 102 via hydride material outlet 204 as a slip stream. In another instance, the hydride material is physically removed (for example, pumped) from the reactor 102 at the end of the reaction period via hydride material outlet 204. To produce hydridable material from the hydride material, hydride material enters the reactor 202 via hydride material inlet 206. Hydride material outlet 204 and hydride material inlet 206 may, in some instances, be connected via piping. In some instances, the hydride material outlet 204 and inlet 206 are configured to allow the hydride material to be removed from the reactor 102 and fed to the reactor 202 in a continuous manner. In the second reactor 202, the hydride material is regenerated. As previously described for System 100, second reactor 202, may be heated to produce hydridable material and hydrogen. The hydrogen may exit the second reactor 202 via a hydrogen conduit 208. In instances, when oxidant is used to regenerate the hydride material, oxidant can enter the second reactor 202 via an oxidant inlet 210. Water, produced from the reaction of the hydride material with the oxidant, can exit the second reactor via a water outlet 212. The regenerated hydridable material can exit the second reactor 202 via a hydridable material outlet 214. The hydridable material may be stored for future use and/or fed into the first reactor 102 via the hydridable material inlet 216. Hydridable material outlet 214 and hydridable inlet 216 may, in some instances, be connected via piping so that the hydridable material may be fed to the first reactor 102 upon demand. As with System 100, the inlets and outlets of the reactors 102 and 202 may be automated to allow feed and hydridable material to be provided to the reactor 102 and hydride material and, if necessary oxidant to be provided to reactor 202 based on the composition of the streams exiting and entering the reactors 102 and 202.

### D. Processing Conditions

The reaction processing conditions in the reactor 102, the reactor 202, or both can be varied to achieve a desired result (e.g., C₂ or higher hydrocarbons and/or hydridable material production). The processing conditions include temperature, pressure, hydrocarbon feed flow, hydridable material flow and/or charge, oxidant flow, or any combination thereof. Processing conditions are controlled, in some instances, to produce products with specific properties. Temperature may range from about 175 to 600° C., 200 to 575° C., 225 to 550° C., or 300 to 400° C. A pressure of 15 to 800 psia (about 0.1 to 5.5 MPa), 50 to 700 psia (about 0.3 to 4.8 MPa), 100 to 500 psi (about 0.7 to 3.4 MPa), or 150 to 450 psia (about 1.0 to 3.1 MPa) in the reactor 102 and/or the reactor 202 can be used. A carrier gas may be combined with the hydrocarbon feed and recirculated through the one or both reactors. Non-limiting carrier gases include nitrogen, helium, argon, or any combination thereof. The carrier gas may enhance mixing in the reactor. Carrier gas can also include any gases used in aiding transfer of hydridable material or hydride material to and from the reactors or hydrocarbons entering the reactors. Severity of the process conditions may be manipulated by changing flow rates of various feed streams and/or carrier streams, the temperature and pressures of the process, feed or carrier gas pre-heat temperature, contact time, or combinations thereof.

### EXAMPLES

The present invention will be described in greater detail by way of specific examples. The following examples are offered for illustrative purposes only, and are not intended to limit the invention in any manner. Those of skill in the art will readily recognize a variety of noncritical parameters which can be changed or modified to yield essentially the same results.

### Prophetic Example 1

### Preparation of C₂ and Higher Hydrocarbons

Production of Ethane.

A stoichiometric amount of hydridable material (Ti metal, 48 g, 1 mole) based on the amount of methane to be treated (28 g, 2 moles) is placed in a stainless steel reactor and heated under flowing dry methane at a temperature of 175 to 600° C. and a pressure of 15 to 800 psia. The gas composition can be monitored by mass spectrometry. The production of ethane can be seen to initiate at temperature at which the TiH₂ material is formed.

### Prophetic Example 2

### Conversion of Alkanes to Alkenes

Production of Propene.

A stoichiometric amount of hydridable material (Ti metal, 48 g, 1 mole) based on the amount of propane to be treated (44 g, 1 moles) is placed in a stainless steel reactor and heated under flowing dry methane at a temperature of 175 to 600° C. and a pressure of 15 to 800 psia. The gas composition can be monitored by mass spectrometry. The production of ethane can be seen to initiate at temperature at which the TiH₂ material is formed.

1. A method for making C₂ or larger hydrocarbons from methane, the method comprising contacting methane with a hydridable material under reaction conditions sufficient to effect removal of at least one hydrogen atom from a plurality of methane molecules to produce a plurality of methyl radicals, wherein the plurality of methyl radicals combine together to form C₂ or larger hydrocarbons, and wherein the reaction is performed in the absence of oxygen gas and reactive metal oxides.

2. The method of claim 1, wherein ethane is produced and the overall reaction is represented by reaction (I):
2 CH₄+XM→C₂H₆+XMH_{y},
where 0.05≧X≦1, M is the hydridable material, and y is 2/X.

3. The method of claim 1, wherein ethylene is produced and the overall reaction is represented by reaction (II):
2 CH₄+XM→C₂H₄+XMH_{y}
where 1≧X≦4, M is the hydridable material, and y is 4/X.

4. The method of claim 1, wherein the formation energy of the hydridable material to the hydride is −101 to −244 kJ/mol (H₂).

5. The method of claim 4, wherein the hydridable material is a metal, a metal alloy, or an intermetallic compound.

6. The method of claim 1, wherein the hydridable material is Li, Na, K, Rb, Cs, Mg, Ca, Sr, Ba, Sc, Y, Ti, Zr, Hf, V, Ta, La, Ce, Th, U, or Gd, preferably Ti, Zr, or Hf, and more preferably Ti.

7. The method of claim 6, wherein a second metal is deposited on the surface of the hydridable material.

8. The method of claim 7, wherein the second metal is a Group IIA metal, Pd, Pt, Ni, or any combination thereof.

9. The method of claim 8, wherein Pd is deposited on the surface of Ti.

10. The method of claim 5, wherein the hydridable material is a metal alloy comprising a Group IIA metal, a Group IVB metal, a Group VIII metal, or a Group IVA.

11. The method of claim 1, wherein the overall reaction is exothermic.

12. The method of claim 1, wherein the reaction conditions include a temperature of 175° C. to 600° C. and a pressure of 15 psia to 800 psia.

13. The method of claim 1, wherein the reaction is performed in the absence of oxygen.

14. The method of claim 1, wherein the hydridable material is supported by a support, preferably a non-reactive metal oxide support.

15. The method of claim 1, wherein the reaction is a stoichiometric or sub-stoichiometric reaction.

16. The method of claim 1, wherein the reaction is performed in a first reactor, the produced hydride is regenerated into the hydridable material, and the regenerated hydridable material is used to react with the methane.

17. The method of claim 1, wherein the produced hydride is subjected to a sufficient amount of heat to regenerate the hydridable material or the produced hydride is subjected to an oxidizing agent to regenerate the hydridable material.

18. The method of claim 1, wherein the reaction is performed in the first reactor and the regeneration of produced hydride to the regenerated hydridable material is performed in a second reactor.

19. The method of claim 1, wherein the reaction is performed in the first reactor and the regeneration of produced hydride to the regenerated hydridable material is performed in the first reactor.

20. A method for removing hydrogen from a hydrocarbon, the method comprising contacting a hydrocarbon with a hydridable material under reaction conditions sufficient to effect removal of at least one hydrogen atom from the hydrocarbon by the hydridable material and form a hydride from the hydridable material and the removed hydrogen atom, wherein the reaction is performed in the absence of oxygen gas and reactive metal oxides.

21. The method of claim 20, wherein the hydrocarbon is a C₂-C₄ hydrocarbon and the reaction is represented by reaction (III):
CₙH₂ₙ₊₂+XM→CₙH_{2n+2-y}+XMH_{y/X},
where
n is from 2 to 4,
y is 2 or 4,
X is ≧1,
M is the hydridable material.

22. The method of claim 21, where n is 2 or 3, y is 2, and X is 1 or 2.

23. The method of claim 21, where n is 4, and y is 2 or 4, and X is 1, 2, or 4.

24. The method of claim 20, wherein the hydrocarbon is methane and the overall reaction is represented by reaction (II):
2 CH₄+XM→C₂H₄+XMH_{y},
where 1≧X≦4, M is the hydridable material, and y is 4/X.

25. The method of claim 22, wherein the formation energy of the hydridable material to the hydride is −64 to −244 kJ/mol (H₂) or −101 to −244 kJ/mol (H₂).

26. The method of claim 26, wherein the hydridable material is a metal, a metal alloy, or an intermetallic compound.

27. The method of claim 20, wherein the hydridable material is Li, Na, K, Rb, Cs, Mg, Ca, Sr, Ba, Sc, Y, Ti, Zr, Hf, V, Ta, La, Ce, Th, U, or Gd, preferably Ti, Zr, or Hf, or more preferably Ti.

28. The method of claim 27, wherein a second metal is deposited on the surface of the hydridable material, wherein the second metal is a Group IIA metal, Pd, Pt, Ni, or a combination thereof.

29. The method of claim 28, wherein Pd is deposited on the surface of Ti.
